(19) United States
(12) Patent Application Publication
(10) Pub. No.: US 2024/0035963 A1
(43) Pub. Date: Feb. 1, 2024

(54) IDENTIFICATION OF CARPET MATERIALS WITH NEAR-INFRARED SPECTROSCOPY

(71) Applicant: CONSENSUS RESOURCE GROUP LTD., Bolton (CA)

(72) Inventors: Albert LI, Mississauga (CA); Tim BEAM, Palgrave (CA)

(21) Appl. No.: 18/379,062

(22) Filed: Oct. 11, 2023

Related U.S. Application Data
(63) Continuation-in-part of application No. 17/525,404, filed on Nov. 12, 2021, now abandoned.

(51) Int. Cl. G01N 21/359; G01N 21/3563; G01N 21/84

(52) U.S. Cl. CPC G01N 21/359; G01N 21/3563; G01N 21/84; G01N 2021/8444; G06Q 40/08

(57) ABSTRACT

A method for identifying carpet materials includes receiving, by a controller coupled to an NIR spectrometer, a predetermined number of NIR measurements of a sample of the carpet material conducted over a bandwidth of a subset of a full NIR spectrum. The NIR spectrometer performs the NIR measurements under control of the controller. Also, sending the NTR measurements to a remote identification server including a spectra library of known carpet materials. The method also includes receiving a matching result from the remote identification server, sending the matching result to a remote appraisal server, and receiving an appraised value of the carpet material.

### CROSS-REFERENCE TO RELATED APPLICATIONS

The present application is a Continuation-in-Part of U.S. application Ser. No. 17/525,404, filed Nov. 12, 2021. The foregoing applications are incorporated by reference herein in their entirety.

### FIELD OF THE INVENTION

The present invention pertains to the field of near-infrared (NIR) spectroscopy, and in particular to a distributed system for identifying carpet materials with NIR spectroscopy.

### BACKGROUND

Current technology is able to identify carpet materials by scanning a physical sample of the carpet with a wideband NIR spectrometer and comparing the collected spectra to a spectra library of known carpet materials. The technology is widely used in the insurance industry to estimate the repair and replacement costs for property losses or damages including carpets. Agents, on behalf of insurance companies, will obtain a carpet sample and send it to a laboratory for analysis. The purpose of identifying the carpet materials of the damaged carpets is to find the best available match for any repairs or replacements, and to provide accurate cost estimates for such repairs or replacements.

The NIR spectroscopy region extends from approximately 780 nm to 2500 nm in the electromagnetic spectrum and a lab NIR spectrometer is able to cover that range. Different materials absorb NIR energy at different wavelengths, so when radiation is absorbed, the NIR spectrometer measures the overtones and combinations of the fundamental molecular vibrational transitions. The absorption wavelengths and/or the corresponding frequencies will form a unique NIR signature based on the chemical and physical properties of the carpet materials being tested.

However, it is not always possible or preferable to take a physical sample of the carpet from the field, and send the physical sample to a lab facility where an NIR spectrometer is used to evaluate and identify the types of carpet materials. This may delay the insurance appraisal process, increase the associated cost, and have a negative impact on the environment. Furthermore, a lab NIR spectrometer equipped with the identification server is not easily portable. It is also prohibitively expensive, difficult to source, and requires customization. To be proficient in operating such an NIR spectrometer would require additional training and experience.

There exists a need for an NIR analysis system that may be used in the field and provide quick, accurate results, that overcomes the limitations of the prior art.

This background information is provided to reveal information believed by the applicant to be of possible relevance to the present invention. No admission is necessarily intended, nor should be construed, that any of the preceding information constitutes prior art against the present invention.

### SUMMARY

An object of the present invention is to provide methods, apparatus, and system for identifying carpet materials with NIR spectroscopy, which can be reliably performed by an untrained person in the field using portable spectrometer devices, managed by a mobile application accessing a remote identification server.

In accordance with an aspect of the present invention, there is provided an apparatus for identifying a carpet material. The apparatus includes an NIR spectrometer, configured to receive an analysis request, and a controller coupled to the NIR spectrometer. The controller controls the NIR spectrometer to perform an analysis of a sample of the carpet material. The analysis includes receiving, from the NIR spectrometer, in response to the analysis request, a batch of a predetermined number of NIR measurements of the sample conducted over a target bandwidth of a subset of a full NIR spectrum. Each of the predetermined number of NIR measurements includes a predetermined number of discrete measurements performed at wavelengths that are evenly distributed across the target bandwidth. Then the controller may send the batch of NIR measurements to a remote identification server including a spectra library of known carpet materials, and receive a matching result from the remote identification server. Upon determining a subset of the batch of NIR measurements are outliers, another batch of the predetermined number of NIR measurements of the sample is scanned over the target bandwidth.

Further embodiments comprise sending the matching result to a remote appraisal server and, in response to sending the matching result, receiving an appraised value of the carpet material generated by the remote appraisal server.

In further embodiments, the controller provides instructions for a user to perform gathering of additional data of the carpet material including a weight of a portion of the carpet material, a length of a pile of the carpet material, and one or more photos of the carpet material. The analysis further includes the controller receiving the additional information and sending the additional data to the remote appraisal server, where the appraised value is based on the matching result and the additional data.

In further embodiments, the identification server and the appraisal server are located remotely from the NIR spectrometer and the controller.

In further embodiments, the controller comprises a mobile application configured to perform any of the functions of controlling the NIR spectrometer to calibrate devices, perform measurements, sending an identification request to the identification server to generate a matching result, sending an appraisal request to the appraisal server to generate an appraisal result, or providing instructions for the user to gather additional data of the carpet material.

In accordance with an aspect of the present invention, there is provided a method for identifying carpet materials including sending an analysis request to an NIR spectrometer, receiving, by a controller coupled to the NIR spectrometer, a batch of predetermined number of NIR measurements of a sample of the carpet material conducted over a target bandwidth of a subset of a full NIR spectrum. The NIR spectrometer performs the NIR measurements under control of the controller. Each of the predetermined number of NR measurements includes a predetermined number of discrete measurements performed at wavelengths that are evenly distributed across the target bandwidth. The method also includes sending the batch of NIR measurements to a remote identification server including a spectra library of known carpet materials and receiving a matching result from the remote identification server. Upon determining a subset of the batch of NIR measurements are outliers, another batch of the predetermined number of NIR measurements of the sample is scanned over the target bandwidth.

Further embodiments include sending the matching result to a remote appraisal server in response to receiving the matching result, and receiving an appraised value of the carpet material in response to sending the matching result.

Further embodiments include receiving, additional data of the carpet material including a weight of a portion of the carpet material, a length of a pile of the carpet material, and one or more photos of the carpet material, and sending the additional data to the remote appraisal server, where the appraised value is based on the matching result and the additional data.

In accordance with an aspect of the present invention, there is provided a system for identifying a carpet material. The system includes an NIR spectrometer, a controller, and a remote identification server. The NIR spectrometer is configured to receive an analysis request. The controller is coupled to the NIR spectrometer, controlling the NIR spectrometer to perform an analysis of a sample of the carpet material. The analysis includes receiving, from the NIR spectrometer, in response to the analysis request, a batch of a predetermined number of NIR measurements of the sample conducted over a target bandwidth of a subset of a full NTR spectrum. Each of the predetermined number of NTR measurements includes a predetermined number of discrete measurements performed at wavelengths that are evenly distributed across the target bandwidth. The remote identification server receives the batch of NIR measurements sent by the controller. The remote identification server includes a spectra library of known carpet materials. The remote identification server sends a matching result corresponding to the NIR measurements to the controller.

Further embodiments include a remote appraisal server for receiving the matching result from the controller and in response to receiving the matching result, sending an appraised value of the carpet material to the controller.

Although example embodiments are described in reference to carpet materials identification for the insurance industry, a person skilled in the art may apply the methods and apparatus as described by example embodiments herein to other suitable purposes.

### BRIEF DESCRIPTION

Further features and advantages of the present disclosure will become apparent from the following detailed description, taken in combination with the appended drawings, in which:

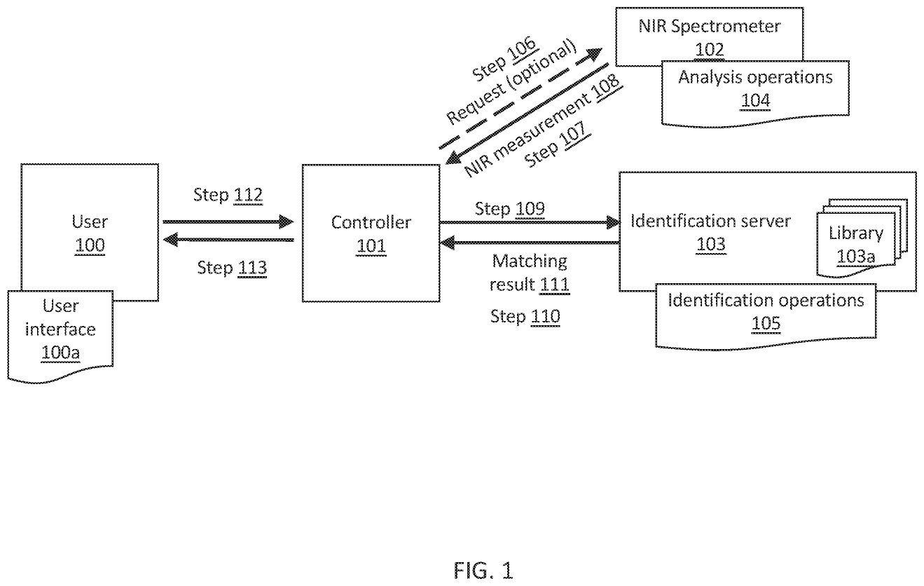

FIG. 1 is a call flow diagram illustrating an operation procedure for a user using a controller, an NIR spectrometer and an identification server in accordance with embodiments.

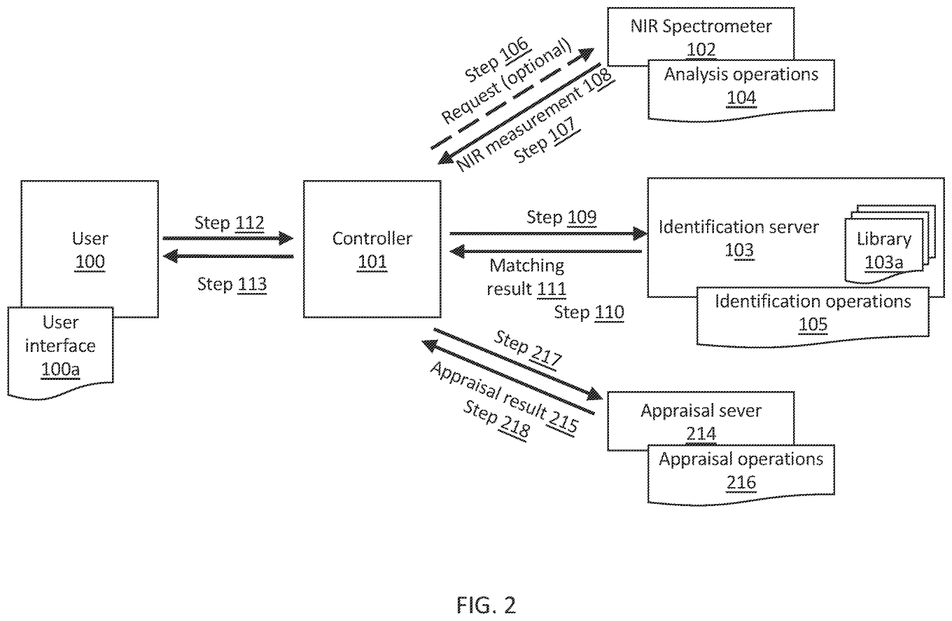

FIG. 2 is a call flow diagram illustrating an operation procedure for a user using a controller, an NIR spectrometer, an identification server, and an appraisal server in accordance with embodiments.

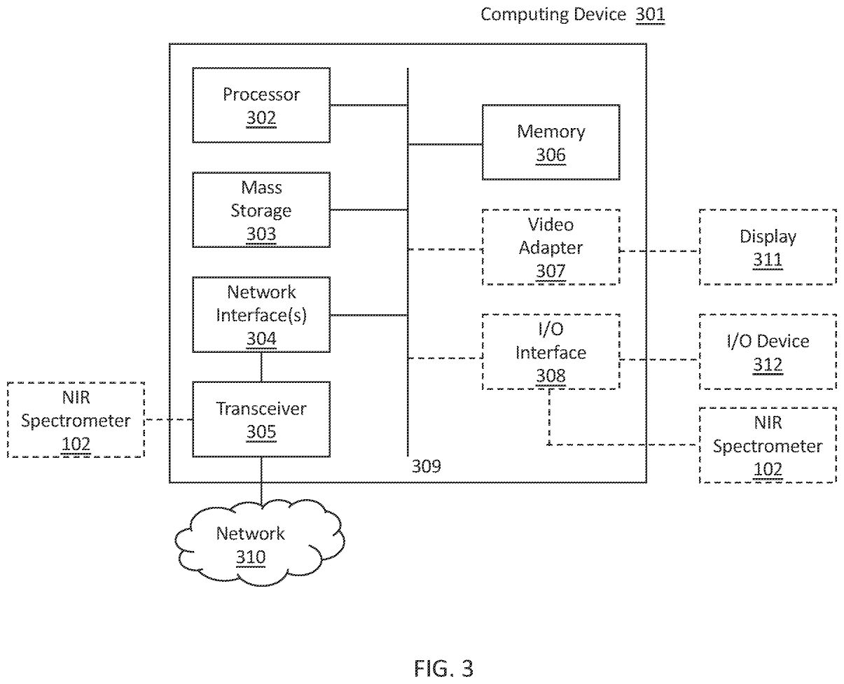

FIG. 3 is a schematic diagram of a controller in accordance with embodiments.

Throughout the appended drawings, like features are identified by like reference numerals.

### DETAILED DESCRIPTION

Embodiments of the present invention include methods, apparatus, and systems for identifying carpet materials using NIR spectroscopy.

An aspect of embodiments is to shorten the long processing, waiting times, and high costs caused by sending physical samples of carpets to a lab facility for analysis. Embodiments may use a controller 101, an NIR spectrometer 102 and a remote identification server 103 to simplify and speed up the process of identifying carpet materials.

In embodiments, the NIR spectrometer 102 is capable of testing using a subset of the entire NIR spectrum, for example, a lower half of the NTR spectrum including a wavelength range of 900 nm to 1700 nm. The NIR spectrometer can measure the overtones and combinations of the fundamental molecular vibrational transitions and the absorption wavelengths or the corresponding frequencies that form a unique NIR signature based on the chemical and physical properties of the carpet materials being tested. In embodiments, the NTR spectrometer 102 may be a portable device, for example, a handheld NIR spectrometer based on a digital micromirror device.

In one embodiment, the NIR spectrometer 102 includes or is coupled with a light source. In embodiments, the light source can be a light-emitting diode (LED) or an array of LEDs. In other embodiments, the light source can employ Digital Light Processing (DLP) technology that uses micro-mirrors to shine different parts of the spectrum sequentially (i.e., one after another) onto a single element detector, thereby building or constructing the spectrum.

With reference to FIG. 1, a controller 101 may be coupled to an NIR spectrometer 102 and an identification server 103. In embodiments, the coupling between the controller 101 and the NIR spectrometer 102 may be implemented by a short-range wireless connection, such as Bluetooth, or through a wired connection, such as USB. In embodiments, the controller 101 and NIR spectrometer 102 may be two physical devices or be packaged or housed as a single physical device. The identification server 103 is located at a remote location and is coupled to the controller 101 over the Internet through wired or wireless networking technologies, such as Wi-Fi or Ethernet. It should be understood that the NTR spectrometer 102 may also be coupled to the identification 103 through the controller 101.

In embodiments, a user 100 may perform one or more of the user operations in the controller 101 such as creating a profile or an account, entering inputs, sending requests, receiving results, or the like, through a user interface 100a, via Step 112. The controller 101 may be triggered to forward an NIR analysis request to the NIR spectrometer 102, via Step 106. In response, under control of the controller 101, the NIR spectrometer 102 may perform a predetermined number of analysis operations 104, upon receiving such the NIR analysis request. Alternatively, in an embodiment, the NIR spectrometer 102 may be configured to self-initiate one or more of analysis operations 104 without a request from the controller 101. The analysis operations may generate a predetermined number of resulting NIR measurement 108, and the NIR measurement 108 may be subsequently sent to the controller 101 via Step 107. In embodiments, the NIR measurement 108 may be a predetermined number of NIR measurements of the carpet material conducted over a bandwidth of a subset of the NIR spectrum. In an embodiment, the controller may send five analysis requests to the NIR spectrometer 102. In another embodiment, the controller may send a single analysis request to the NIR spectrometer 102 which may perform five measurements in response to the analysis request from the controller. After receiving the NIR measurement 108, the controller 101 may send the NIR measurement via Step 109 to the remote identification server 103 which comprises one or more network accessible spectra libraries 103a of known carpet materials. The identification server 103 may be configured to perform one or more of identification operations 105, such as comparative analysis by comparing the received NIR measurement with the network accessible spectra library 103a of known carpet materials for highest resemblance or similarity. Accordingly, the identification server 103 may generate a matching result 111, and the matching result 111 may be sent to the controller 101, via Step 110. In embodiments, the identification server can accurately identify the fibre composition of carpet materials commonly used in the manufacture of carpets, including without limitation Linen, Silk, Polyester (PET), Cotton, Nylon 6 (PA6), Nylon 6.6 (PA66), Rayon/Viscose, Polypropylene/Olefin (PP), Triexta (PTT), Wool, and various blends. The user 100 may access any of the information or data stored in or received by the controller 101 via the user interface. For example, the user is able to request and receive the matching result 111 via Step 112 and Step 113. In embodiments, the matching result may be displayed as a composition of 40% cotton and 60% polyester, with or without an accuracy rate. Generally, the process from taking scans of the damaged carpets to receiving the displayed result will be faster than that of the prior art.

In another embodiment, each scan of the damaged carpet sample is conducted by the NIR spectrometer 102 which performs a large amount of point or discrete measurements (e.g., over 200 measurement points for each scan taken). In embodiments, these point or discrete measurements are distributed or taken evenly across the target wavelength range of 900 to 1700 nm which falls within the lower portion of the full NIR spectrum. For instance, a hypothetical sequence of measurement points may commence at 900 nm, followed by 904 nm, 908 nm, and so forth. As a result, in embodiments, the aggregation or compilation of these 200+ measurement points can generate a graphical representation of the target range spectrum for each scan. Due to the large volume of point or discrete measurements for each scan, the graphical representation manifests as a continuous curve or pattern within/across the target wavelength range (e.g., 900 to 1700 nm).

When endeavoring to ascertain the predominant fiber type(s) of a carpet sample, a pre-determined number of NIR scans/measurements (for example, a batch or round of 5 scans) are conducted or performed for each carpet sample. In embodiments, when a round of 5 scans of a carpet sample is taken, and if the analysis algorithm determines that more than 2 scans from this batch or round are outliers or likely to be outliers, the user is prompted to undertake an additional batch or round of 5 scans. In practice, the accuracy rate identifying fiber type is approximately 97.5% when applying the accepted batch or round of 5 scans.

In embodiments, a predetermined number of NIR scan/measurements will generate the same number of graphical representations, and each graphical representation, as described above, can manifest as a continuous curve or pattern within/across the target wavelength range (e.g., 900 to 1700 nm).

In embodiments, the data derived from the scan or multiple scans, along with the measured weight and pile height (other quantitative aspects of the carpet sample) and pre-determined photographs of the sample (other qualitative aspects of the carpet sample) are used to determine a carpet product of like-kind and quality.

In another embodiment, a single scan can reliably determine a single fiber type. With a round of a predetermined number of scans (e.g., a batch of 5 scans), the system can reliably determine the majority fiber type(s) contained in the carpet sample (e.g., up to 5 types of fibers). In practice, the batch of a predetermined number of scans can be any number pre-determined, set/programmed and stored in the system. In embodiments, if required, more than the per-determined number of scans (e.g., more than 5 scans) can be taken or performed to determine multiple fiber compositions. Since modern carpet products are usually highly weighted or concentrated in the primary fiber, identifying the majority fiber type(s) would serve as a sufficient contribution or a determinative factor for the purpose of determining like-kind and quality of the carpet sample, and subsequently appraising the value of the carpet sample (e.g., for insurance purposes).

In another embodiment, a round or batch of 5 scans (or more) is sent to the server and applied to the identification models. For example, if a subset of the total scans (e.g., 3 (or more) of the 5 scans) are acceptable, a fiber identification is returned. If 2 or more scans are determined to be outliers or likely to be outliers based on the algorithm/analysis, then the user is prompted to start another round of 5 new scans, and send again to the server to be applied to the identification models.

In yet another embodiment, for a round or batch of 5 scans (each scan with 200+ measurements), if 2 or more scans are deemed outliers, then the entire batch of 5 scans is discarded, and the user is prompted to procure another 5 scans to be sent as a new batch to the server to be applied to the identification models. Once a majority fiber type or types are determined, the fiber type identification is combined with carpet sample weight, carpet sample pile height, and information derived from the photographs, including style, gauge, primary backing, secondary backing and color composition, to determine carpet products of like-kind and quality.

In embodiments, the controller 101 may collect or assign metadata to the data. Metadata may be received or measured by the controller 101, received from the NIR spectrometer 102, received from the remote identification server 103, or be received from other servers or sensors. The controller 101 may be used to manage the information or data received, stored, or archived, based on one or more of various features, groupings, or categories such as measurement time, location, carpet brand, individual users or companies, etc.

With reference to FIG. 2, a controller 101 may be coupled to an NIR spectrometer 102, a remote identification server 103, and a remote appraisal server 214. In another embodiment, the appraisal server 214 may be configured to be coupled with the identification server 103. The remote appraisal server 214 and the remote identification server 103 may be any form of physical or virtual server and may make use of cloud computing technology. The remote appraisal server 214 and the remote identification server 103 may be the same physical or virtual server unit or may be separate and may be located at the same or different locations.

As illustrated in FIG. 2, a user 100 may perform one or more of user operations and either of the NIR spectrometer 102 and the identification server 103 may perform one or more of the analysis operations 104 and the identification operations 105 respectively under the control of the controller 101, as illustrated in FIG. 1 and aforementioned embodiments.

With reference to FIG. 2, the controller may further send the matching result 111 via Step 217 to a remote appraisal server 214 which is configured to perform one or more of appraisal operations 216. The remote appraisal server 214 may comprise one or more of pricing libraries of known carpet materials. In one embodiment, the appraisal server 214 may generate an appraisal result 215 based on the matching result 111. In another embodiment, the appraisal server 214 may generate an appraisal result 215 based on the matching result 111 together with any additional data gathered and entered into the controller 101 by the user 100 via the user interface 100a. Such additional data of the carpet material may include one or more of the following: a weight of a portion of the carpet material, a length of a pile of the carpet material, and a photo of the carpet material.

Furthermore, the appraisal result 215 may comprise a single value or a range of values and the value may be accompanied with a confidence level or an estimate buffer (e.g. ±10%). The appraisal result 215 may be sent to the controller 101, via Step 218.

Accordingly, the user 100 may access any of the information or data stored in or received by the controller 101 via a user interface 100a. For example, the user 100 is able to request and receive the appraisal result 215 via Step 112 and Step 113. In embodiments, the appraisal result 215 may be displayed together with additional information such as suggested replacement brands, carpet suppliers or the like.

In embodiments, the controller may comprise a smart phone together with a mobile application which is configured to perform any or all of: controlling the NIR spectrometer to calibrate the device, perform measurements, controlling the identification server to generate a matching result, controlling the appraisal server to generate an appraisal result, or providing instructions for the user to gather additional data of the carpet material. The mobile application may be compatible with various types of controller system operating systems including iOS and Android. In embodiments, the identification server or the appraisal server may be implemented on a cloud server.

In embodiments, a user (e.g., a layperson, or a person with no specialized training or experience) may reliably perform the aforementioned steps and operations in the field using portable spectrometer devices, managed by a mobile application accessing a remote identification server or both a remote identification server and a remote appraisal server.

FIG. 3 is a schematic diagram of a computing device 301 that may be used as a controller 101. A controller may perform any or all of operations of the above methods and features explicitly or implicitly described herein, according to different embodiments of the present invention. For example, a computer or a mobile phone equipped with network function may be configured as a computing device 301. In embodiments, device 301 may optionally include NIR spectrometer 102 housed within. In embodiment, NIR spectrometer 102 may be a physically separate device coupled to device 301.

As illustrated in FIG. 3, the computing device 301 includes a processor 302, such as a central processing unit (CPU) or specialized processors such as a graphics processing unit (GPU) or other such processor unit, memory 306, non-transitory mass storage 303, I/O interface 308, network interface 304, video adaptor 307, and a transceiver 305, all of which are communicatively coupled via bi-directional bus 309. Video adapter 307 may be connected to one or more of display 311 and I/O interface 308 may be connected to one or more of I/O devices 312 which may be used to implement a user interface. According to embodiments, any or all of the depicted elements may be utilized, or only a subset of the elements. Further, the computing device 301 may contain multiple instances of certain elements, such as multiple processors, memories, or transceivers. Also, elements of the hardware device may be directly coupled to other elements without the bi-directional bus. Additionally, or alternatively to a processor and memory, other electronics, such as integrated circuits, may be employed for performing the required logical operations.

The memory 306 may include any type of non-transitory memory such as static random access memory (SRAM), dynamic random access memory (DRAM), synchronous DRAM (SDRAM), read-only memory (ROM), any combination of such, or the like. The mass storage element 303 may include any type of non-transitory storage device, such as a solid state drive, hard disk drive, a magnetic disk drive, an optical disk drive, USB drive, or any computer program product configured to store data and machine executable program code. According to embodiments, the memory 306 or mass storage 303 may have recorded thereon statements and instructions executable by the processor 302 for performing any of the aforementioned method operations described above.

It will be appreciated that, although specific embodiments of the technology have been described herein for purposes of illustration, various modifications may be made without departing from the scope of the technology. The specification and drawings are, accordingly, to be regarded simply as an illustration of the invention as defined by the appended claims, and are contemplated to cover any and all modifications, variations, combinations, or equivalents that fall within the scope of the present invention. In particular, it is within the scope of the technology to provide a computer program product or program element, or a program storage or memory device such as a magnetic or optical wire, tape or disc, or the like, for storing signals readable by a machine, for controlling the operation of a computer according to the method of the technology and/or to structure some or all of its components in accordance with the system of the technology.

Acts associated with the method described herein can be implemented as coded instructions in a computer program product. In other words, the computer program product is a computer-readable medium upon which software code is recorded to execute the method when the computer program product is loaded into memory and executed on the microprocessor of the wireless communication device.

Further, software-related operations of the method may be executed on any computing device, such as a personal computer, server, PDA, or the like and pursuant to one or more, or a part of one or more, program elements, modules or objects generated from any programming language, such as C++, Java, Python, or the like. In addition, each operation, or a file or object or the like implementing each said operation, may be executed by special purpose hardware or a circuit module designed for that purpose.

Through the descriptions of the preceding embodiments, the present invention may be implemented by using hardware only or by using software and a necessary universal hardware platform. Based on such understandings, the technical solution of the present invention may be embodied in the form of a software product. The software product may be stored in a non-volatile or non-transitory storage medium, which can be a compact disk read-only memory (CD-ROM), USB flash disk, or a removable hard disk. The software product includes a number of instructions that enable a computer device (personal computer, server, or network device) to execute the methods provided in the embodiments of the present invention. For example, such an execution may correspond to a simulation of the logical operations as described herein. The software product may additionally or alternatively include number of instructions that enable a computer device to execute operations for configuring or programming a digital logic apparatus in accordance with embodiments of the present invention.

Embodiments have been described above in conjunctions with aspects of the present disclosure upon which they can be implemented. Those skilled in the art will appreciate that embodiments may be implemented in conjunction with the aspect with which they are described but may also be implemented with other embodiments of that aspect. When embodiments are mutually exclusive, or are otherwise incompatible with each other, it will be apparent to those skilled in the art. Some embodiments may be described in relation to one aspect, but may also be applicable to other aspects, as will be apparent to those of skill in the art.

1. An apparatus for identifying a carpet material, the apparatus comprising:
an NIR spectrometer, configured to receive an analysis request; and
a controller coupled to the NIR spectrometer, the controller controlling the NIR spectrometer to perform an analysis of a sample of the carpet material, the analysis including:
receiving, from the NIR spectrometer, in response to the analysis request, a batch of a predetermined number of NIR measurements of the sample scanned over a target bandwidth of a lower-half subset of a full NIR spectrum, each of the predetermined number of NIR measurements including a predetermined number of discrete measurements performed at wavelengths that are evenly distributed across the target bandwidth;
sending the batch of NIR measurements to a remote identification server including a spectra library of known carpet materials; and
receiving in response to sending the batch of NIR measurements to the remote identification server, a matching result generated by the remote identification server;
wherein upon determining a subset of the batch of NIR measurements are outliers, another batch of the predetermined number of NIR measurements of the sample is scanned over the target bandwidth.

2. The apparatus of claim 1, wherein the analysis further comprises sending the matching result to a remote appraisal server and, in response to sending the matching result, receiving an appraised value of the carpet material generated by the remote appraisal server.

3. The apparatus of claim 2, wherein the controller provides instructions for a user to perform gathering of additional data of the carpet material including a weight of a portion of the carpet material, a length of a pile of the carpet material, and a photo of the carpet material;
the analysis further including the controller receiving the additional information and sending the additional data to the remote appraisal server, wherein the appraised value is based on the matching result and the additional data.

4. The apparatus of any of claim 2 wherein the identification server and the appraisal server are located remotely from the NIR spectrometer and the controller.

5. The apparatus of any of claim 2 wherein the controller comprises a mobile application configured to perform any of the functions of:
controlling the NIR spectrometer to perform measurements;
sending an identification request to the identification server to generate a matching result;
sending an appraisal request to the appraisal server to generate an appraisal result; or
providing instructions for the user to gather additional data of the carpet material.

6. A method for identifying carpet materials comprising:
sending, to an NIR spectrometer, an analysis request;
receiving, by a controller coupled to the NIR spectrometer, in response to the analysis request, a batch of a predetermined number of NIR measurements of a sample of the carpet material scanned over a target bandwidth of a lower-half subset of a full NIR spectrum, the NIR spectrometer performing the NIR measurements under control of the controller, each of the predetermined number of NIR measurements including a predetermined number of discrete measurements performed at wavelengths that are evenly distributed across the target bandwidth;
sending the batch of NIR measurements to a remote identification server including a spectra library of known carpet materials; and
receiving in response to sending the batch of NIR measurements to the remote identification server, a matching result generated by the remote identification server;
wherein upon determining a subset of the batch of NIR measurements are outliers, another batch of the predetermined number of NIR measurements of the sample is scanned over the target bandwidth.

7. The method of claim 6 further comprising:
sending, in response to receiving the matching result, the matching result to a remote appraisal server; and
receiving, in response to sending the matching result, a appraised value of the carpet material generated by the remote appraisal server.

8. The method of claim 7 further comprising:
receiving, additional data of the carpet material including a weight of a portion of the carpet material, a length of a pile of the carpet material, and a photo of the carpet material; and
sending the additional data to the remote appraisal server, wherein the appraised value is based on the matching result and the additional data.

9. A system for identifying a carpet material, the system comprising:
an NIR spectrometer, configured to receive an analysis request;
a controller coupled to the NIR spectrometer, the controller controlling the NIR spectrometer to perform an analysis of a sample of the carpet material, the analysis including receiving, from the NIR spectrometer, in response to the analysis request, a batch of a predetermined number of NIR measurements of the sample scanned over a target bandwidth of a lower-half subset of a full NIR spectrum, each of the predetermined number of NIR measurements including a predetermined number of discrete measurements performed at wavelengths that are evenly distributed across the target bandwidth; and
a remote identification server for receiving the batch of NIR measurements sent by the controller, the remote identification server including a spectra library of known carpet materials, the remote identification server generating and sending, in response to receiving the batch of NIR measurements, a matching result corresponding to the batch of NIR measurements to the controller.

10. The system of claim 9 further comprising a remote appraisal server for receiving the matching result from the controller and, in response to receiving the matching result, sending an appraised value of the carpet material generated by the remote appraisal server to the controller.

11. The apparatus of claim 1, wherein the lower-half subset of a full NIR spectrum includes a wavelength range of 900 nm to 1700 nm.

12. The apparatus of claim 1, wherein the predetermined number of NIR measurements of the sample is five.
